(19) United States
(12) Patent Application Publication
(10) Pub. No.: US 2025/0241601 A1
(43) Pub. Date: Jul. 31, 2025

(54) METHOD AND COMPUTING DEVICE USING MACHINE LEARNING FOR PULMONARY ARTERY PRESSURE MEASUREMENT BASED ON ELECTRICAL IMPEDENCE TOMOGRAPHY

(71) Applicant: DISPLAID INC., MONTREAL (CA)

(72) Inventors: Carlos Cesar FALLAQUE BERTRAN, Laval (CA); Jonathan BOUDREAU, SAINT-JEAN-SUR-RICHELIEU (CA); Mathieu RIVET, DELSON (CA); Frederick DROUIN, MONTREAL (CA)

(73) Assignee: DISPLAID INC., MONTREAL (CA)

(21) Appl. No.: 19/039,824

(22) Filed: Jan. 29, 2025

Related U.S. Application Data
(60) Provisional application No. 63/626,729, filed on Jan. 30, 2024.

(51) Int. Cl. A61B 5/00; A61B 5/0205; A61B 5/021; A61B 5/0265; A61B 5/0536; A61B 5/08; G16H 10/60; G16H 40/63

(52) U.S. Cl. CPC A61B 5/7278; A61B 5/0205; A61B 5/0536; A61B 5/6823; A61B 5/6831; A61B 5/7267; A61B 5/7275; G16H 10/60; G16H 40/63; A61B 5/02125; A61B 5/0265; A61B 5/08

(57) ABSTRACT

Computing device and method using machine learning for pulmonary artery pressure measurement based on electrical impedance tomography. The following steps are repeated n times: (i) inject an alternating electrical current between a pair of electrodes, the pair of electrodes being located on a belt, the belt being positioned around the chest of a person; (ii) determine corresponding voltage values between m pairs of electrodes located on the belt. The determined n*m voltage values are transmitted to the computing device. The computing device executes a machine learning engine, the machine learning engine using a predictive model stored at the computing device for inferring outputs based on inputs. The inputs comprise the determined n*m voltage values. The outputs comprise a matrix of values representative of at least one of: lung perfusion, pulse transit time values and pulmonary artery pressure values.

### TECHNICAL FIELD

The present disclosure relates to the field of Electrical Impedance Tomography (EIT). More specifically, the present disclosure relates to a method and computing device using machine learning for pulmonary artery pressure (PAP) measurement based on electrical impedance tomography.

### BACKGROUND

One application of Electrical Impedance Tomography (EIT) is the measurement of PAP of the patient's lung perfusion. Based on data generated by EIT, a tomographic reconstruction is performed, for visually illustrating location(s) in the lungs where the perfusion is present. From this region of interest, it is possible to derive the pulse transit time (PTT), the time for the blood to reach the lungs from the heart. The PTT is inversely proportional to the PAP.

EIT is based on the injection of a current or voltage pattern through the skin of a patient. The present disclosure specifically addresses current-mode EIT, where a current is injected. Reconstruction of an electrical bio-impedance distribution is performed, based on voltages collected by electrodes positioned on the skin of the patient. Typically, the patient wears a belt comprising electrodes for injecting the current pattern, as well as electrodes for collecting the resulting voltages.

The usage of EIT for PAP measurement relies on the fact that different internal body materials have different electrical bio-impedances. For example, the electrical bio-impedance of extracellular water present in the lungs is different from the electrical bio-impedance of intracellular water present in the cells of the lungs. The voltages collected through EIT depend on the electrical bio-impedance of the body materials present in the lungs and traversed by the injected current.

One technique for measuring PAP using EIT is the usage of a mathematical model. The mathematical model is used to generate a visual representation of the location(s) where lung perfusion is present, based on the collected voltages, and to derive PTT and PAP values. However, mathematical models are very sensible to noise. Consequently, the detection of small electrical bio-impedance variations, representative of the presence of lung perfusion, is difficult.

In various technological fields, the usage of machine learning techniques, such as neural networks, has proven to be an effective replacement of traditionally used algorithms for improving accuracy and/or reliability.

There is therefore a need for a new method and computing device using machine learning for PAP measurement based on EIT.

### SUMMARY

According to a first aspect, the present disclosure relates to a method using machine learning for pulmonary artery pressure (PAP) measurement based on electrical impedance tomography. The method comprises repeating the following steps n times: (i) injecting an alternating electrical current between a pair of electrodes, the pair of electrodes being located on a belt, the belt being positioned around the chest of a person, (ii) determining corresponding voltage values between m pairs of electrodes located on the belt. The method comprises transmitting the determined n*m voltage values to a computing device. The method comprises receiving by the computing device the determined n*m voltage values. The method comprises executing by the computing device a machine learning engine. The machine learning engine uses a predictive model stored at the computing device for inferring outputs based on inputs. The inputs comprise the determined n*m voltage values. The outputs comprise a matrix of values representative of at least one of: lung perfusion, pulse transit time (PTT) values and PAP values.

In a particular aspect, the determination of the corresponding voltage values is performed between m pairs of adjacent electrodes located on the belt.

In another particular aspect, the m pairs of electrodes used for determining the corresponding voltage values do not include the electrodes of the pair of electrodes used for injecting the alternating electrical current.

In yet another particular aspect, the inputs of the machine learning engine further comprise at least one of the following: a frequency of the alternating electrical current, an electrical current value of the alternating electrical current and a voltage matrix of electrocardiogram (ECG) values.

In another particular aspect, the alternating electrical current has a sinusoidal waveform.

In still another particular aspect, the voltage values comprise average voltage values, Root Mean Square (RMS) voltage values or maximum values of alternating voltage waveforms measured between the pairs of adjacent electrodes.

In yet another particular aspect, the machine learning engine is a neural network inference engine implementing a neural network, the neural network using the predictive model for inferring the outputs based on the inputs, the predictive model comprising weights of the neural network. In an embodiment, the neural network comprises an input layer, followed by fully connected hidden layers, followed by an output layer. The input layer comprises neurons receiving the inputs. The output layer comprises neurons outputting the outputs. The weights of the neural network are applied to the fully connected hidden layers.

According to a second aspect, the present disclosure relates to a computing device. The computing device comprises a communication interface, memory storing a predictive model, and a processing unit comprising one or more processors. The processing unit is configured to receive, via the communication interface, a plurality of voltage values between pairs of electrodes of a belt comprising a plurality of electrodes. The voltage values are determined by sequentially injecting an alternating electrical current between different pairs of electrodes of the belt, the belt being positioned around the chest of a person. The processing unit is further configured to execute a machine learning engine. The machine learning engine uses a predictive model stored at the computing device for inferring outputs based on inputs. The inputs comprise the plurality of voltage values. The outputs comprise a matrix of values representative of at least one of: the lung perfusion, the PTT values and the PAP values.

### BRIEF DESCRIPTION OF THE DRAWINGS

Embodiments of the disclosure will be described by way of example only with reference to the accompanying drawings, in which:

FIG. 1 provides a schematic representation of an Electrical Impedance Tomography (EIT) system;

FIGS. 2A and 2B illustrate a schematic view of a transverse sectional view of the chest of a person with respective left and right lungs;

FIG. 3 provides a schematic representation of a computing device adapted for processing voltage values determined by the EIT system of FIG. 1 to perform lung perfusion, PTT and/or PAP measurements;

FIG. 4 illustrates a method using machine learning for lung perfusion, PTT and/or PAP measurements based on EIT;

FIG. 5 provides a schematic representation of a machine learning engine executed by the computing device of FIG. 3; and

FIGS. 6 and 7 represent respective exemplary configurations of a neural network implementing the machine learning engine of FIG. 5.

### DETAILED DESCRIPTION

The foregoing and other features will become more apparent upon reading of the following non-restrictive description of illustrative embodiments thereof, given by way of example only with reference to the accompanying drawings. Like numerals represent like features on the various drawings.

Various aspects of the present disclosure generally address the problem of measuring pulmonary artery pressure (PAP) using Electrical Impedance Tomography (EIT). More specifically, the present disclosure aims at using machine learning techniques for processing voltage values generated by the injection of an electrical current according to the EIT technique.

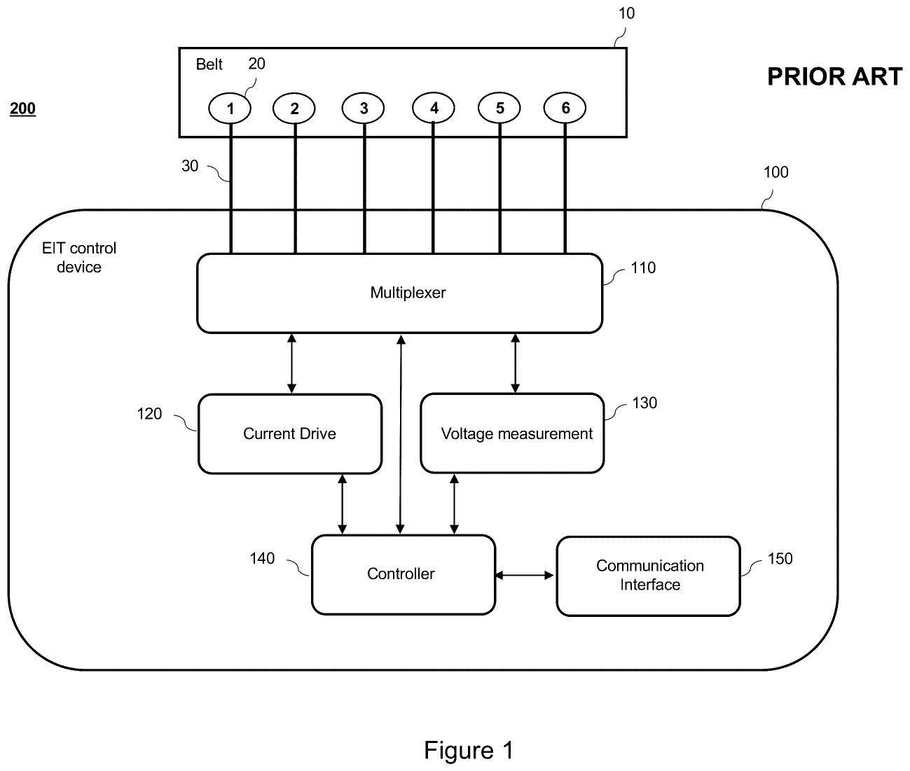

Referring now to FIG. 1, a schematic representation of an Electrical Impedance Tomography (EIT) system 200 is provided. The EIT system 200 comprises a belt 10 with electrodes 20 and an EIT control device 100. EIT systems such as the one illustrated in FIG. 1 are well known in the art. The implementation of the EIT system 200 illustrated in FIG. 1 is for illustration purposes only. A person skilled in the art would readily understand that other implementations are supported by the present disclosure.

The belt 10 comprises a plurality of electrodes 20. In the rest of the description, the number of electrodes 20 will be referred to as N, N being an integer. FIG. 1 represents the belt 10 having six electrodes 20 only for simplification purposes. Any number N of electrodes 20 is within the scope of the present disclosure. For example, configurations with 16 or 32 electrodes attached to the belt 10 are typically used. An electrode 20 at position on the belt 10 will be referred to as electrode (i), i varying from 1 to N.

The belt 10 is positioned around the chest of a person (not represented in FIG. 1 for simplification purposes). A sinusoidal electrical current (an alternating current (AC) with a sinusoidal waveform) is injected between two electrodes. A corresponding voltage is measured between several adjacent electrodes. The measured voltages are generated by a circulation of the injected electrical current within the body of the person. In the context of the present disclosure, the belt 10 is positioned around the chest of the person, to generate a current circulation within the lungs of the person.

The present disclosure is not limited to the injection of a sinusoidal electrical current. Other types of alternating currents having a waveform different from a sinusoid are also within the scope of the present disclosure.

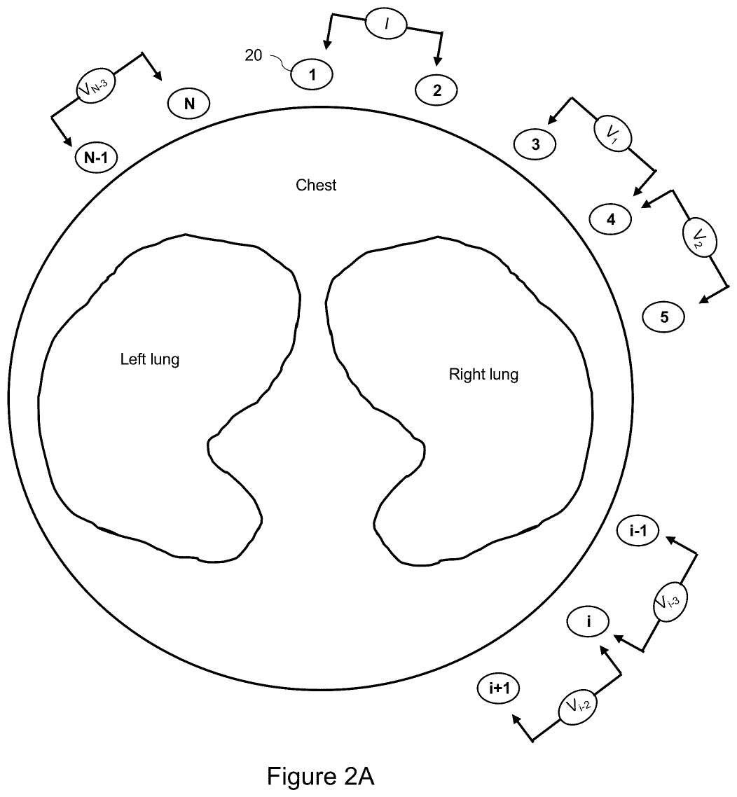

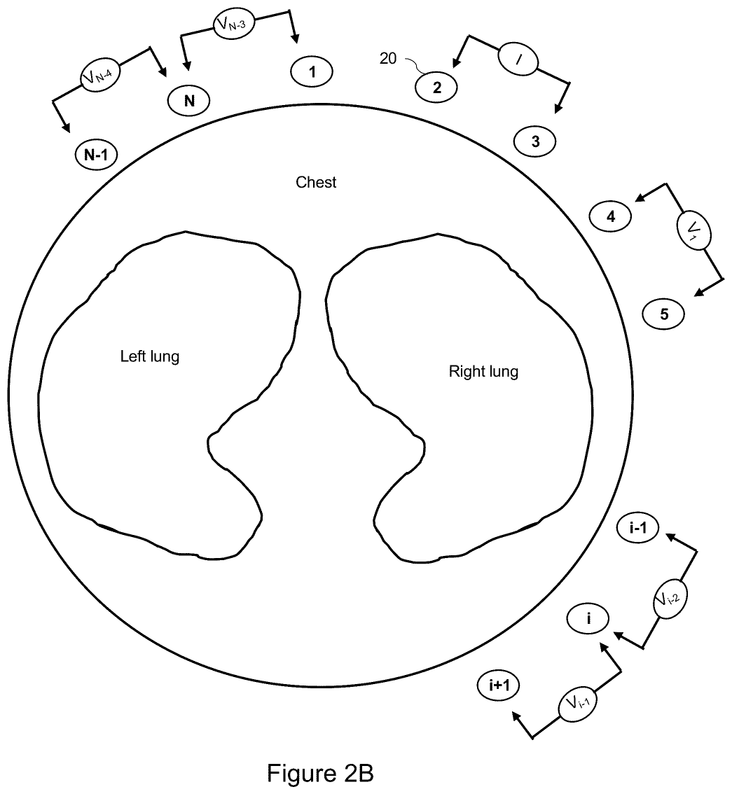

For example, the sinusoidal electrical current is injected between electrodes (1) and (2), and voltages are measured respectively between electrodes (3) and (4), and electrodes (5) and (6). Then, the sinusoidal electrical current is injected between electrodes (2) and (3), and voltages are measured respectively between electrodes (4) and (5), and electrodes (6) and (1). Then, the sinusoidal electrical current is injected between electrodes (3) and (4), and voltages are measured respectively between electrodes (5) and (6), and electrodes (1) and (2). The pattern for injecting the sinusoidal electrical current and measuring the corresponding voltages for a belt 10 having N electrodes 20 will be detailed later in relation to FIGS. 2A and 2B.

The EIT control device 100 controls the injection of the sinusoidal electrical current and the measurement of the corresponding voltages.

The EIT control device 100 comprises a multiplexer circuit 110 which is connected to the N electrodes 20 of the belt 10. For example, each electrode 20 is connected to the multiplexer circuit 110 via an electrical cable 30. The multiplexer circuit 110 is controlled by a controller 140; and is connected to a current drive circuit 120 and a voltage measurement circuit 130.

The current drive circuit 120 generates the sinusoidal electrical current that is injected between two electrodes 20 via the multiplexer circuit 110.

The voltage measurement circuit 130 performs the voltage measurement between two electrodes 20 via the multiplexer circuit 110.

The controller 140 controls the current drive circuit 120, the voltage measurement circuit 130, and the multiplexer circuit 110. For example, the controller 140 triggers the current drive circuit 120 to generate a sinusoidal electrical current with given characteristics (e.g. amplitude, frequency, etc.). The controller 140 further configures the multiplexer circuit 110 to inject the sinusoidal electrical current between electrodes (1) and (2). Then, the controller 140 triggers the voltage measurement circuit 130 to perform voltage measurements. The controller 140 further configures the multiplexer circuit 110 to perform a first voltage measurement between electrodes (3) and (4). Then, the controller 140 configures the multiplexer circuit 110 to perform a second voltage measurement between electrodes (5) and (6).

Exemplary implementations of the current drive circuit 120, the voltage measurement circuit 130, the multiplexer circuit 110 and the controller 140 are well known in the art (and are out of the scope of the present disclosure). In particular, the controller 140 generally comprises a microcontroller (or another type of integrated circuit chip) with a processor, memory, input/output peripherals, etc.

The EIT control device 100 also comprises a communication interface 150. The communication interface 150 allows the reception of commands, which are processed by the controller 140 for controlling operations of the current drive circuit 120, the voltage measurement circuit 130, the multiplexer circuit 110. The communication interface 150 allows the transmission of data, for example the voltage measurements performed under the control of the controller 140. Examples of communication interfaces include a Wi-Fi communication interface, a Bluetooth® communication interface, etc.

Reference is now made concurrently to FIGS. 1, 2A and 2B, where FIGS. 2A and 2B represent schematic views of a transverse sectional view of the chest of a person with respective left and right lungs.

The belt 10 of FIG. 1 is positioned around the chest of the person. The belt 10 comprises N electrodes 20. For simplification purposes, only electrode numbers (1), (2), (3), (4), (5) . . . (i-1), (i), (i+1) . . . (N-1) and (N) are represented in FIGS. 2A and 2B.

The EIT system 200 illustrated in FIG. 1 is used for performing the voltage measurements. The belt 10, electrical cables 30 and EIT control device 100 are not represented in FIGS. 2A and 2B for simplification purposes. The voltage measurements are performed by repeating the following two steps: inject an alternating electrical current between a pair of injecting electrodes among the N electrodes of the belt 10 and perform corresponding voltage measurements between pairs of measuring electrodes among the N electrodes of the belt 10. The two steps are repeated iteratively by selecting the pair of injecting electrodes according to a first pattern and performing the voltage measurements between the pairs of measuring electrodes selected according to a second pattern. In general, for a given pair of injecting electrodes, the pairs of measuring electrodes do not include the electrodes from the given pair of injecting electrodes. The iteration is repeated n times, n depending on the first pattern. Each iteration results in a number m of voltage measurements depending on the second pattern. Thus, a total of n*m voltage measurements is generated. Alternatively, the number of determined voltage values is not the same for each iteration.

Following is a first exemplary sequence of voltage measurements comprising N iterations, performed under the control of the controller 140 of the EIT control device 100.

Iteration 1 is illustrated in FIG. 2A: a sinusoidal electrical current I is injected between adjacent electrodes (1) and (2). N-3 voltage measurements are performed between adjacent pairs of electrodes, excluding electrodes (1) and (2). FIG. 2A illustrates voltage measurement V₁ between electrodes (3) and (4), voltage measurement V₂ between electrodes (4) and (5), voltage measurement Vᵢ₋₃ between electrodes (i-1) and (i), voltage measurement Vᵢ₋₂ between electrodes (i) and (i+1), and voltage measurement V_{N-3} between electrodes (N-1) and (N). The other voltage measurements are not represented in FIG. 2A for simplification purposes.

Iteration 2 is illustrated in FIG. 2B: the sinusoidal electrical current I is injected between adjacent electrodes (2) and (3). N-3 voltage measurements are performed between adjacent pairs of electrodes, excluding electrodes (2) and (3). FIG. 2B illustrates voltage measurement V₁ between electrodes (4) and (5), voltage measurement Vᵢ₋₂ between electrodes (i-1) and (i), voltage measurement Vᵢ₋₁ between electrodes (i) and (i+1), voltage measurement V_{N-4} between electrodes (N-1) and (N), and voltage measurement V_{N-3} between electrodes (N) and (1). The other voltage measurements are not represented in FIG. 2B for simplification purposes.

Although not represented in the Figures for simplification purposes, a person skilled in the art would readily understand how the next iterations are performed, by performing a clockwise rotation of the electrodes of injection of the sinusoidal electrical current I.

The belt 10 having N electrodes, a total of N*(N−3) voltage measurements is generated by performing N iterations of the current injection and voltage measurement patterns.

The sequence of voltage measurements can be adapted by performing a counterclockwise instead of a clockwise rotation of the electrodes of injection of the sinusoidal electrical current I.

The injected electrical current I being sinusoidal, the voltage measured between a pair of adjacent electrodes varies over time. For example, the voltage also has a sinusoidal waveform. More generally, the voltage has an alternating voltage waveform. Over a period of time T, the voltage measurement circuit 130 measures a plurality of instantaneous voltage values of the voltage between the pair of adjacent electrodes. The plurality of instantaneous voltage values is transmitted to the controller 140. The controller 140 determines a voltage value representative of the voltage between the pair of adjacent electrodes. The representative voltage value is calculated based on the plurality of instantaneous voltage values. For example, the controller 140 calculates an average voltage value, a Root Mean Square (RMS) voltage value, a maximum voltage value, etc.

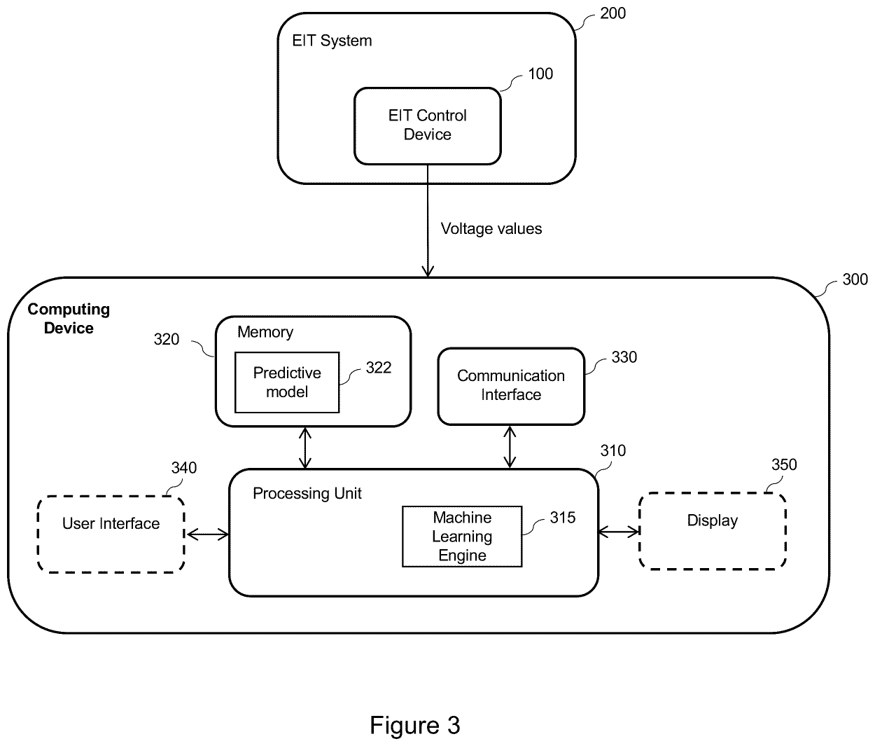

At the end of the sequence of voltage measurements, the controller 140 has determined N*(N−3) voltage values. The N*(N−3) voltage values are transmitted via the communication interface 150 to a computing device 300 represented in FIG. 3.

Following are alternative exemplary sequences of voltage measurements performed under the control of the controller 140 of the EIT control device 100.

Instead of injecting the sinusoidal electrical current I between adjacent electrodes, the sinusoidal electrical current I is injected between two electrodes i and i+x, x being an integer greater than one (the first exemplary sequence corresponds to x=1). Following is a pattern of injection similar to the one described in the first exemplary sequence, with x=3. For illustration purposes, the number of electrodes N is set to 16. The following sequence of current injection is performed: between electrodes 1 and 4, then 2 and 5, then 6 and 9, then 7 and 10, then 8 and 11, then 9 and 12, then 10 and 13, then 11 and 14, then 12 and 15, then 13 and 16, then 14 and 1, then 15 and 2, then 16 and 3.

For each current injection between a pair of electrodes i and i+x, the voltage measurements are performed between adjacent pairs of electrodes, excluding electrodes i and i+x (the pattern of voltage measurement is similar to the pattern of voltage measurement described in the first exemplary sequence). Alternatively, the voltage measurements are not performed between pairs of adjacent electrodes, but between pairs of electrodes j and j+y, y being an integer greater than one. In another alternative, the voltage measurements are performed between pairs of electrodes j and j+z, z being an integer taking several values for a given value of j.

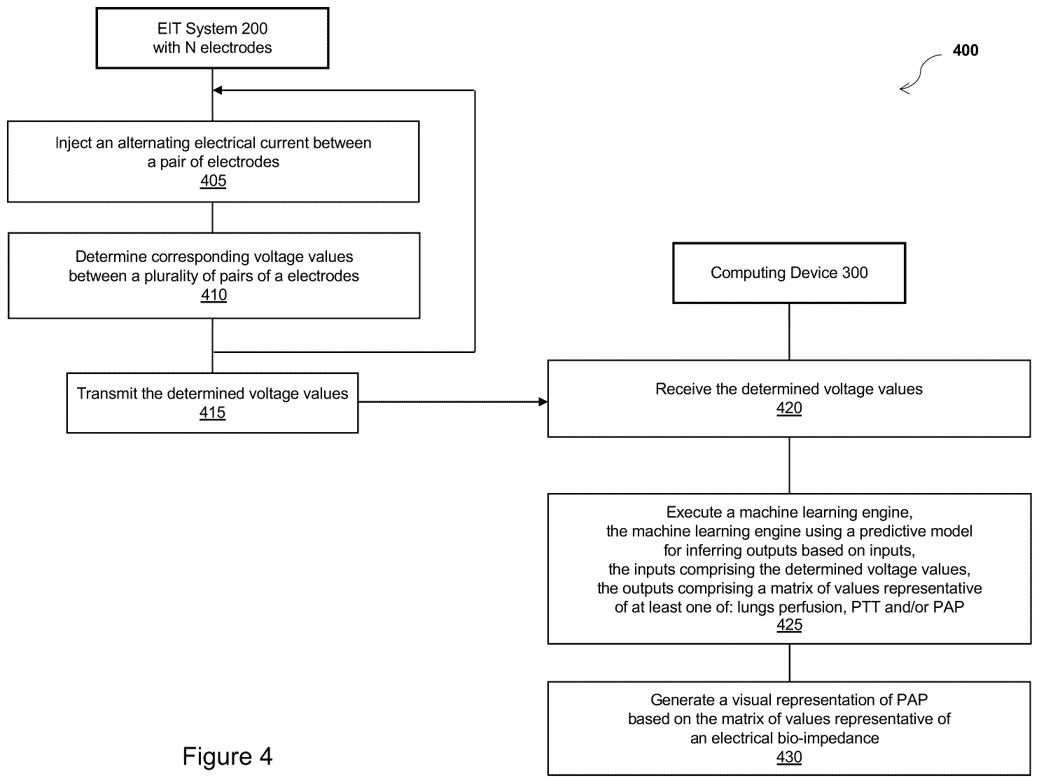

A person skilled in the art would readily understand that the patterns of current injection and the patterns of voltage measurement are not limited to those described in the present disclosure. However, any of these patterns result in the determination of corresponding voltage values, which are processed according to the method 400 illustrated in FIG. 4.

Referring now to FIG. 3, a schematic representation of the computing device 300 is provided. The EIT system 200 illustrated in FIG. 3 corresponds to the EIT system 200 of FIG. 1 (only the EIT control device 100 is represented for simplification purposes).

The computing device 300 comprises a processing unit 310, memory 320, and at least one communication interface 330. The computing device 300 may comprise additional components, such as a user interface 340, a display 350, etc.

The processing unit 310 comprises one or more processors (not represented in FIG. 3 for simplification purposes) capable of executing instructions of a computer program. Each processor may further comprise one or several cores. The processing unit 310 may also include one or more dedicated processing components (e.g. an Application Specific Integrated Circuits (ASIC) for performing compute-intensive tasks, etc.). The processing unit 310 executes a machine learning engine 315, which will be described later in the description.

The memory 320 stores instructions of computer program(s) executed by the processing unit 310, data generated by the execution of the computer program(s) by the processing unit 310, data received via the communication interface(s) 330, etc. The memory 320 stores a predictive model 322 used by the machine learning engine 315, which will be described later in the description. Only a single memory 320 is represented in FIG. 3, but the computing device 300 may comprise several types of memories, including volatile memory (such as Random Access Memory (RAM)) and non-volatile memory (such as a hard drive, Erasable Programmable Read-Only Memory (EPROM), Electrically-Erasable Programmable Read-Only Memory (EEPROM), etc.).

The communication interface 330 allows the computing device 300 to exchange data with other devices (e.g. the EIT control device 100). Examples of communication interfaces 330 of the wireline type include standard (electrical) Ethernet ports, fiber optic ports, ports adapted for receiving Small Form-factor Pluggable (SFP) units, etc. The communication interface 330 may also be of the wireless type (e.g. a Wi-Fi interface, a Bluetooth® interface, etc.). The communication interface 330 comprises a combination of hardware and software executed by the hardware, for implementing the communication functionalities of the communication interface 330. Alternatively, the combination of hardware and software for implementing the communication functionalities of the communication interface 330 is at least partially included in the processing unit 310.

Referring now concurrently to FIGS. 1, 3 and 4, a method 400 using machine learning for lung perfusion, PTT and/or PAP based on EIT is represented in FIG. 4. At least some of the steps of the method 400 are performed by the EIT system 200 illustrated in FIG. 1 and at least some of the steps of the method 400 are performed by the computing device 300 illustrated in FIG. 3. The method 400 is applied to the belt 10 comprising the N electrodes 20, the belt 10 being positioned around the chest of a person.

The method 400 comprises the step 405 of injecting an alternating electrical current between a pair of electrodes of the belt 10. Step 405 is executed by components of the EIT control device 100, including the controller 140, the current drive circuit 120 and the multiplexer circuit 110.

The method 400 comprises the step 410 of determining corresponding voltage values between a plurality of pairs of electrodes 20 of the belt 10. Step 410 is executed by components of the EIT control device 100, including the controller 140, the voltage measurement circuit 130 and the multiplexer circuit 110.

Steps 405 and 410 are repeated n times according to a pattern of current injection, as described previously. Furthermore, each execution of step 410 results in the generation of m voltage values, according to a pattern of voltage measurement, as described previously. Thus, after the n iterations of steps 405 and 410, a total of n*m voltage values has been determined. For example, in the first exemplary sequence of voltage measurements described previously in relation to FIGS. 2A and 2B, a total of N*(N−3) voltage values are determined. Alternatively, the number of voltage values determined at step 410 is not the same for each iteration of steps 405 and 410.

From the perspective of the computing device 300, the EIT system 200 is seen as a black box transmitting a plurality of voltage values determined between pairs of electrodes 20 of the belt 10. The voltage values are determined by sequentially injecting the alternating electrical current between different pairs of electrodes 20 of the belt 10, according to the pattern of current injection and the pattern of voltage measurement.

The method 400 comprises the step 415 of transmitting the determined voltage values to the computing device 300 via the communication interface 150 of the EIT control device 100. Step 415 is executed by the controller 140 of the computing device 100.

The method 400 comprises the step 420 of receiving the determined voltage values via the communication interface 330 of the computing device 300. Step 420 is executed by the processing unit 310 of the computing device 300.

The method 400 comprises the step 425 of executing the machine learning engine 315. The machine learning engine 315 uses the predictive model 322 (stored in the memory 320 of the computing device 300) for inferring outputs based on inputs. The inputs comprise the determined voltage values. The outputs comprise a matrix of values representative of at least one of the lung perfusion, the PTT values and the PAP values. Step 425 is executed by the processing unit 310 of the computing device 300.

The method 400 comprises the step 430 of generating a visual representation of at least one a lung perfusion, PTT and/or PAP (determined at step 425). Step 430 is executed by the processing unit 310 of the computing device 300.

The implementation of step 430 is dependent on the outputs inferred at step 425 by the machine learning engine 315.

In a first exemplary implementation, the matrix of values representative of lung perfusion, PTT and/or PAP is a matrix of pixel values. For example, each pixel value comprises three components consisting of Red Green Blue (RGB) values. The matrix is a 2-dimensonal (2-D) array, the two dimensions representing a coordinate system (e.g. a cartesian coordinate system). Each pixel value of the matrix comprises the three components encoding the RGB values. In another example, each pixel value consists of a single component representative of a gray-scale image. Step 430 is implemented by displaying on the display 350 of the computing device 300 an image corresponding to the matrix of pixel values. Alternatively or complementarily, the matrix of pixel values is transmitted to another computing device (not represented in the Figures for simplification purposes), the other computing device displaying (on its own display) an image corresponding to the matrix of pixel values. The displayed image is a representation of a portion of the lungs, the lung perfusion, PTT and PAP being represented by pixels having a pre-defined color (or set of colors).

In a second exemplary implementation, the matrix of values representative of lung perfusion, PTT and/or PAP is processed by one or more algorithms, to generate a corresponding matrix of pixel values. As mentioned previously, an image corresponding to the matrix of pixel values is then displayed (e.g. on the display 350 of the computing device 300).

At step 425 of the method 400, additional parameter(s) may be used as input(s) of the machine learning engine 315. For example, the frequency of the alternating electrical current injected at step 405 is used as input. Alternatively, or complementarily, an electrical current value of the alternating electrical current injected at step 405 is used as input. Alternatively, or complementarily, a voltage matrix containing electrocardiogram (ECG) values at step 405 is used as input. Examples of the electrical current value include an average electrical current value, a Root Mean Square (RMS) electrical current value, a maximum electrical current value, etc. In an exemplary implementation, the additional parameters are transmitted by the controller 140 via the communication interface 150 of the EIT control device 100; and received by the processing unit 310 via the communication interface 330 of the computing device 300.

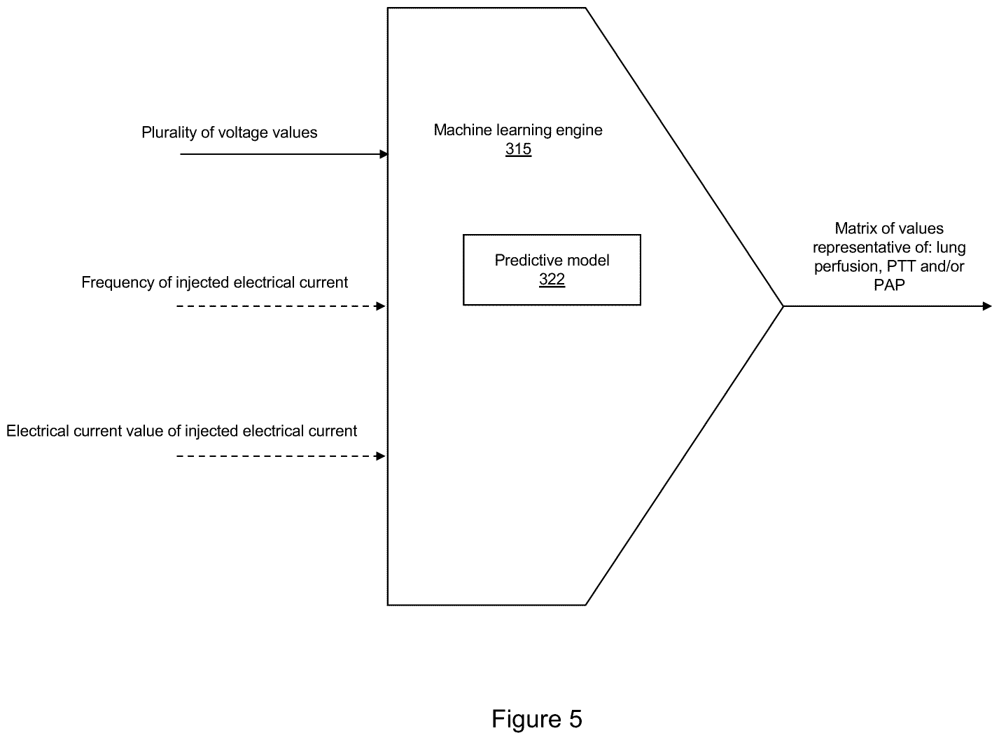

Reference is now made concurrently to FIGS. 3, 4 and 5. FIG. 5 illustrates the inputs and the outputs used by the machine learning engine 315 when performing step 425.

The inputs include the plurality of voltage values determined by the EIT system 200 and transmitted to the computing device 300. Optional parameters used as inputs (e.g. the previously mentioned frequency of the injected alternating electrical current and/or electrical current value of the injected alternating electrical current and/or a voltage matrix containing electrocardiogram (ECG) values) are also represented.

The plurality of voltage values used as inputs can be represented as a matrix. For example, when the n iterations of steps 405 and 410 generate m voltage values at each iteration, the plurality of voltage values is represented as a matrix of n*m values.

The outputs include the matrix of values representative of at least one of the lung perfusion, the PTT values and the PAP values.

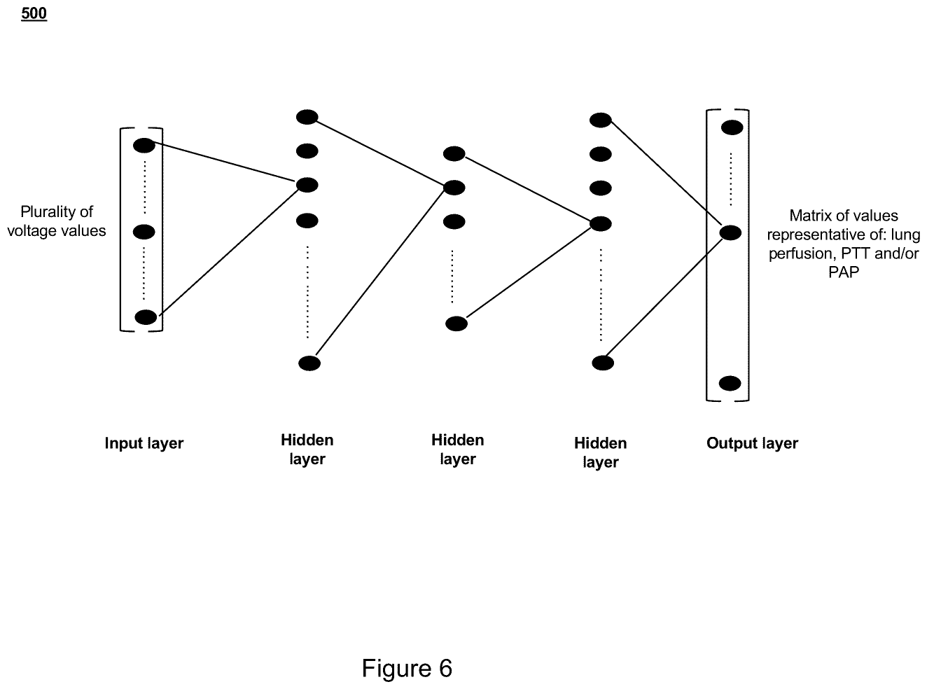

Reference is now made concurrently to FIGS. 3, 4, 5 and 6. FIG. 6 illustrates the machine learning engine 315 being implemented by a neural network 500.

The neural network 500 comprises an input layer, followed by one or more intermediate hidden layers, followed by an output layer, where the hidden layers are fully connected. The input layer comprises one neuron for receiving each one of the plurality of voltage values determined by the EIT system 200 and transmitted to the computing device 300. The input layer comprises additional neurons (not represented in FIG. 6) if additional input parameter(s) are used (e.g. the optional input parameter(s) illustrated in FIG. 5). The number of hidden layer(s) is an integer greater or equal than 1 (FIG. 6 represents three hidden layers for illustration purposes only). The number of neurons in each hidden layer may vary. During the training phase of the neural network 500, the number of hidden layers and the number of neurons for each hidden layer are selected (and may be adapted experimentally). The output layer comprises one neuron for outputting each one of the values of the lung matrix of values representative of at least one of the lung perfusion, the PTT values and the PAP values.

A layer L being fully connected means that each neuron of layer L receives inputs from every neuron of layer L-1 and applies respective weights to the received inputs. By default, the output layer is fully connected to the last hidden layer.

The generation of the outputs based on the inputs using weights allocated to the neurons of the neural network 500 is well known in the art for a neural network using only fully connected hidden layers. The architecture of the neural network 500, where each neuron of a layer (except for the first layer) is connected to all the neurons of the previous layer is also well known in the art. The weights allocated to the neurons are part of the predictive model 322 illustrated in FIG. 5.

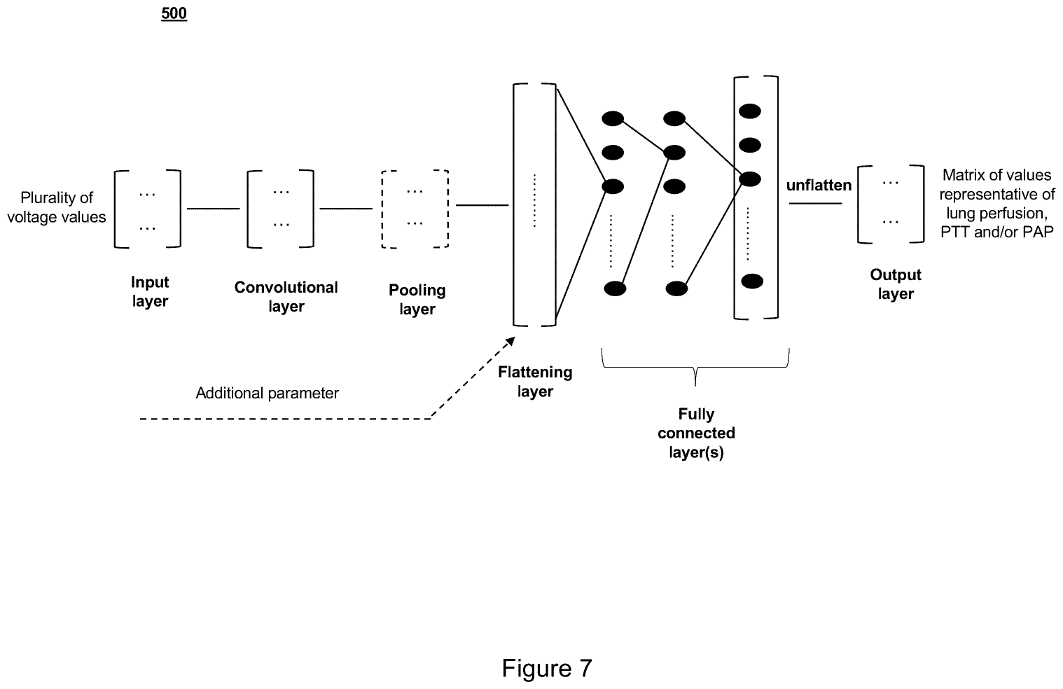

Reference is now made concurrently to FIGS. 3, 4, 5 and 7. FIG. 7 illustrates the machine learning engine 315 being implemented by a neural network 500 using convolution layer(s).

The neural network 500 includes an input layer for receiving the plurality of voltage values determined by the EIT system 200 in the form of a matrix.

The input layer is followed by a convolutional layer (e.g. a two-dimension (2D) convolutional layer) which generates a convoluted matrix. As is well known in the art, the convolutional layer is defined by the following parameters: a filter and a stride.

The convolutional layer is optionally followed by a pooling layer, which generates a pooled matrix. As is well known in the art, the pooling layer is defined by the following parameters: a filter, a stride, and a pooling algorithm. The role of the pooling layer is to reduce the size of the matrix generated by the convolutional layer.

The convolutional layer (or the pooling layer if it is present) is followed by a flattening layer. As is well known in the art, the flattening layer converts the matrix of the convolutional layer (or pooling layer) into a one-dimensional (1 D) matrix.

The flattening layer is followed by one or more fully connected layers. The operations of the fully connected layer(s) have been described previously in relation to FIG. 6.

The one or more fully connected layer is followed by an output layer for outputting the matrix of values representative of at least one of the lung perfusion, the PTT values and the PAP values. As illustrated in FIG. 7, the last layer of the fully connected layer(s) is un-flattened, to convert the 1D matrix of the last fully connected layer into the matrix of the output layer (which may be a 2D or 3D matrix).

Although a single convolution layer (and a corresponding optional pooling layer) is represented in FIG. 7, several consecutive convolution layers (and corresponding optional pooling layers) may be included in the neural network 500, as is well known in the art.

As illustrated in FIG. 7, any additional parameter (e.g. the frequency of the injected alternating electrical current and/or the electrical current value of the injected alternating electrical current and/or a voltage matrix containing electrocardiogram (ECG) values) is not processed by the convolutional layer(s), but directly injected in the flattening layer, as is well known in the art.

The present disclosure is not limited to the configurations of neural networks illustrated in FIGS. 6 and 7. The present disclosure supports any configuration capable of receiving the plurality of voltage values as inputs and generating the matrix of values representative of at least one of: the lung perfusion, the PTT values and the PAP values as outputs. Furthermore, other machine learning techniques (such as a regression analysis algorithm, a Bayesian network algorithm, a K-nearest neighbor (KNN) algorithm, a K means algorithm, etc.) may be used in place of a neural network for implementing the machine learning engine 315.

Referring back to FIGS. 5 and 6, a training phase for generating the predictive model 322 will now be described. The training phase is described in the context of the neural network 500 illustrated in FIG. 6. However, the implementation of a training phase is well known in the art of machine learning, and the following training phase can be adapted to other configurations of the neural network 500. Furthermore, the following training phase can also be adapted to other machine learning techniques.

A training engine (not represented in the Figures) implementing the neural network 500 illustrated in FIG. 6 is used during the training phase. The inputs and the outputs used by the training engine are the same as those previously described for the machine learning engine 315. The training phase consists in generating the predictive model 322 that is used during an operational phase by the machine learning engine 315. The predictive model 322 includes the following characteristics of the neural network 500: the number of layers, the number of neurons per layer, and the weights associated to the neurons of the fully connected hidden layers. The values of the weights are automatically calculated during the training phase. Furthermore, during the training phase, the number of layers and the number of neurons per layer can be adjusted to improve the accuracy of the model.

Various techniques well known in the art of neural networks are used for performing (and improving) the generation of the predictive model 322, such as forward and backward propagation, usage of bias in addition to the weights (bias and weights are generally collectively referred to as weights in the neural network terminology), supervised learning unsupervised learning, reinforcement leaning, etc.

The training phase is generally based on the generation of a training set comprising multiple instances of training inputs and corresponding expected outputs of the neural network 500. The training engine adapts the weights of the neural network 500, so that when presented with instances of the training inputs, the corresponding outputs generated by the neural network 500 match the expected outputs (generally with a pre-defined tolerance margin).

In an exemplary implementation, the expected outputs are generated with one or more legacy mathematical models (not based on machine learning techniques) performing tomographic reconstruction based on the training inputs. The training inputs are selected in conditions where noise can be avoided, so that the training phase is not subject to the sensibility to noise of the mathematical models. Furthermore, a specialist in the art of tomographic reconstruction can review the expected outputs generated with the mathematical model(s) and improve the accuracy of the expected outputs.

In another exemplary implementation, the training phase further includes training the neural network to detect, compensate and/or eliminate measurements from an improperly placed electrode or for an electrode that is not properly maintained around the chest for the duration of the sequence of voltage measurements. By training the neural network to detect, compensate and/or eliminate measurements from for an improperly placed electrode or for an electrode that is not maintained around the chest for the duration of the sequence of voltage measurements, the resulting neural network becomes more resilient and prevents from having to repeat a sequence of voltage measurements.

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. A method using machine learning for pulmonary artery pressure measurement based on electrical impedance tomography (EIT), the method comprising:
repeating the following steps n times:
injecting an alternating electrical current between a pair of electrodes, the pair of electrodes being located on a belt, the belt being positioned around the chest of a person; and
determining corresponding voltage values between m pairs of electrodes located on the belt;
transmitting the determined n*m voltage values to a computing device;
receiving by the computing device the determined n*m voltage values; and
executing by the computing device a machine learning engine, the machine learning engine using a predictive model stored at the computing device for inferring outputs based on inputs, the inputs comprising the determined n*m voltage values, the outputs comprising a matrix of values representative of at least one of: lung perfusion, pulse transit time values and pulmonary artery pressure values.

2. The method of claim 1, wherein the determination of the corresponding voltage values is performed between m pairs of adjacent electrodes located on the belt.

3. The method of claim 1, wherein the m pairs of electrodes used for determining the corresponding voltage values do not include the electrodes of the pair of electrodes used for injecting the alternating electrical current.

4. The method of claim 1, wherein the inputs of the machine learning engine further comprise at least one of the following: a frequency of the alternating electrical current, an electrical current value of the alternating electrical current and a voltage matrix containing electrocardiogram (ECG) values.

5. The method of claim 1, wherein the alternating electrical current has a sinusoidal waveform.

6. The method of claim 1, wherein the voltage values comprise average voltage values, Root Mean Square (RMS) voltage values or maximum values of alternating voltage waveforms measured between the pairs of adjacent electrodes.

7. The method of claim 1, wherein the machine learning engine is a neural network inference engine implementing a neural network, the neural network using the predictive model for inferring the outputs based on the inputs, the predictive model comprising weights of the neural network.

8. The method of claim 7, wherein the neural network comprises an input layer, followed by fully connected hidden layers, followed by an output layer; the input layer comprising neurons receiving the inputs; the output layer comprising neurons outputting the outputs; the weights of the neural network being applied to the fully connected hidden layers.

9. A computing device comprising:
a communication interface;
memory storing a predictive model; and
a processing unit comprising one or more processors configured to:
receive via the communication interface a plurality of voltage values between pairs of electrodes of a belt comprising a plurality of electrodes, the voltage values being determined by sequentially injecting an alternating electrical current between different pairs of electrodes of the belt, the belt being positioned around the chest of a person;
execute a machine learning engine, the machine learning engine using a predictive model stored at the computing device for inferring outputs based on inputs, the inputs comprising the plurality of voltage values, the outputs comprising a matrix of values representative of at least one of lung perfusion values, pulse transit time (PTT) values and the pulmonary artery pressure (PAP) values.

10. The computing device of claim 9, wherein the processing unit further generates a visual representation of a lung perfusion array based on the matrix of at least one of values representative of the lung perfusion, the PTT values and the PAP values.

11. The computing device of claim 10, wherein generating a visual representation comprises applying at least one algorithm to the matrix of values representative of at least one of: the lung perfusion, the PTT values and the PAP values to generate an array of lung perfusion values, and further displaying a trend corresponding to the matrix of at least one of: the lung perfusion, the PTT values and the PAP values on a display of the computing device.

12. The computing device of claim 9, wherein the inputs of the machine learning engine further comprise at least one of the following: a frequency of the alternating electrical current, an electrical current value of the alternating electrical current and a voltage matrix containing electrocardiogram (ECG) values.

13. The computing device of claim 9, wherein the machine learning engine is a neural network inference engine implementing a neural network, the neural network using the predictive model for inferring the outputs based on the inputs, the predictive model comprising weights of the neural network.

14. The computing device of claim 10, wherein the neural network comprises an input layer, followed by fully connected hidden layers, followed by an output layer; the input layer comprising neurons receiving the inputs; the output layer comprising neurons outputting the outputs; the weights of the neural network being applied to the fully connected hidden layers.
